(19) United States
(12) Patent Application Publication
(10) Pub. No.: US 2026/0091110 A1
(43) Pub. Date: Apr. 2, 2026

(54) NOVEL SURFACTANTS AND USES THEREOF

(71) Applicant: Hoffmann-La Roche Inc., Little Falls, NJ (US)

(72) Inventors: Mirjam Clemens-Hemmelmann, Lörrach (CH); Lisa Dietel, Basel (CH); Hannah Sophia Kientz, Offenburg (CH)

(73) Assignee: Hoffmann-La Roche Inc., Little Falls, NJ (US)

(21) Appl. No.: 19/336,318

(22) Filed: Sep. 22, 2025

Related U.S. Application Data
(63) Continuation of application No. PCT/EP2024/057696, filed on Mar. 22, 2024.

(30) Foreign Application Priority Data
Mar. 24, 2023 (EP) 23163888.3

(51) Int. Cl. A61K 39/395; A61K 47/22

(52) U.S. Cl. CPC A61K 39/39591; A61K 47/22

(57) ABSTRACT

The present invention provides aqueous pharmaceutical compositions comprising the compounds of formula (I) as defined herein, as well as novel methods and uses of said compounds as surfactants in aqueous antibody compositions.

### CROSS-REFERENCE TO RELATED APPLICATIONS

This application is continuation of PCT Application No. PCT/EP2024/057696, filed Mar. 22, 2024, which claims the priority of European Application No. 23163888.3, filed Mar. 24, 2023, each of which is incorporated by reference in its entirety.

### FIELD OF THE INVENTION

The present invention relates to the field of aqueous pharmaceutical antibody formulations, which are stabilized against the formation of visible particles comprising, for example, antibody aggregates, aggregates of antibodies and silicon oil or particles based on free fatty acids and the like.

### BACKGROUND OF THE INVENTION

Surfactants are crucial excipients in protein formulations as they protect the labile protein from interfacial stress that may lead to protein aggregation. Proteins, such as monoclonal antibodies (mAb), are administered parenterally, which limits the choice of the surfactant, including one of the most commonly used surfactants polysorbate 20 (PS20), but also polysorbate 80, poloxamer 188, and Kolliphor/Solutol® HS 15 (poly-oxyethylene ester of 12-hydroxystearic acid). PS20 can degrade over the shelf-life of a product either by oxidative degradation or by enzymatic, hydrolytic degradation. In particular, the latter yields free fatty acids (FFA) as degradation products, which can precipitate in solution and subsequently form sub-visible and visible particles. Under conditions typically found in biopharmaceutical formulations, FFA can precipitate even below their solubility limit dependent on temperature but the time point of particle precipitation is poorly understood even for well-characterized degradation profiles.

Therefore, there is a need for alternative surfactants which do not show liabilities regarding intrinsic stability and adsorption behavior to pharmaceutically relevant interfaces. In particular, there remains a need to investigate novel/alternative surfactants to mitigate existing liabilities of established surfactants for parenteral administration in order to expand the toolbox for formulation development while guaranteeing optimal drug product stability.

The present invention solves this problem by providing compounds for the new use as surfactants in aqueous antibody formulations, preferably aqueous compositions of therapeutic, monoclonal antibodies.

### BRIEF DESCRIPTION OF THE FIGURES

FIG. 1: Thermal conformational stability of mAbs in presence of surfactants (only mAb formulations and surfactants with considerable impact of thermal conformation and references are shown). Figures show the average value of two individual measurements of onset temperature (Tₒₙ; left) and melting temperature (Tₘ₁; right). Surfactants with shaded values demonstrate a considerable decrease of thermal stability properties compared to the control formulation without surfactant (w/o) and benchmark surfactants (PS20 and Px188).

FIG. 2: Visible particles of fpMab formulations detected in EP box after 7 days shaking at 5° C. presented in a heat map: class (I): 0 particles, class (II): 1-3 particles in max 1 of 3 vials, class (III) ≥4 particles in 1 or 1-2 particles in 2 of 3 vials and class (IV): ≥2 vials >5 particles or ≥3 vials >2 particles.

FIG. 3: Visible particles detected in EP box after different storage times declared in weeks (w) at different conditions: class (I): 0 particles, class (II): 1-4 particles in max 30% vials, class (III) >5 particles in <30% vials or 1 particles in <50% vials and class (IV): >30% vials >5 particles or >40% vials >2 particles. Classes are marked in different grey intensities (darker grey means higher class). Formulations in pre-fillable syringes (pfs) are marked accordingly.

FIG. 4: Turbidity of mAbs formulations with different storage conditions (39 weeks at 5° C., 26 weeks at 25° C./65% rH, and 13 weeks at 40° C./75% rH compared to initial values) and surfactant concentrations. Turbidity is measured in Nephelometric Turbidity Units (NTU). Only the turbidityat the end of the storage time is shown in the heat map.

FIG. 5: Soluble aggregate levels, given as an increase in HMWs (area %) after different storage conditions and surfactant concentrations (0.06 (1st dot), 0.2 (2nd dot), and 0.6 mg/mL (3rd dot each)): (A) 5° C.; (B) 25° C./60% rH; (C) 40° C./75% rH for 4 weeks ( [symbol] ), 12 weeks ( [symbol] ), 26 weeks ( [symbol] ), and 39 weeks ( [symbol] ) compared to initial values (−).

FIG. 6: Cumulative count of sub-visible particles ≥10 μm per mL for formulations with 0.06, 0.2, and 0.6 mg/mL surfactant. SVP count after storage at 5° C. for 39 weeks, at 25° C./60% rH for 26 weeks, and at 40° C./75% rH for 12 weeks. Darker color represents higher count of SVP.

FIG. 7: Particle characterization of selected formulations by FTIR mainly to check the presence of protein and protein-PDMS particles. Measurements were taken at the last time point of the stability study.

FIG. 8: Selected FlowCam images of protein-PDMS particles (PPP) after 13 weeks storage at 40° C./75% rH in Mab2 formulations with poloxamer.

FIG. 9: Visible particles detected in EP box after one week shaking (sk) and five freeze-thaw cycles (F/T): class (I): 0 particles, class (II): 1-4 particles in max 30% vials, class (III) >5 particles in <30% vials or 1 particles in <50% vials and class (IV): >30% vials >5 particles or >40% vials >2 particles.

FIG. 10: Turbidity of mAbs formulations with different stress conditions (one week shaking (sk) at 5/25° C. and five freeze-thaw cycles (F/T)) and surfactant concentrations. Turbidity is measured in Nephelometric Turbidity Units (NTU). Samples marked with * were not measured because of a very high quantity of particles.

FIG. 11: Cumulative count of sub-visible particles ≥10 μm per mL for formulations with 0.06, 0.2, and 0.6 mg/mL surfactant. SVP count after shaking for 7 days at 5° C., at 25° C./60% rH, and 5 times freeze−thaw cycles −20/5° C. (F/T). * show samples which are not measured because of too many particles (particle limit for method reached).

FIG. 12: Soluble aggregate levels, given as increase in HMW (area %) after different stress conditions (7 days shaking at 5° C., 7 days shaking at 25° C., and 5 freeze/thaw −20/5° C. cycles (F/T)) and surfactant concentrations (0.06, 0.2, and 0.6 mg/mL): bsMab2, IgG4, and MP compared to initial values (first line).

### DETAILED DESCRIPTION OF THE INVENTION

In one embodiment, the present invention provides an aqueous pharmaceutical composition comprising an antibody and a surfactant wherein the surfactant is a compound of formula (I)

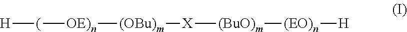

wherein
   —OE is ethoxy;
   —OBu is n-butoxy;
   —X— is —O-n-butylene-O— or

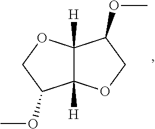

n is 40, 48 or 68 and
m is 11, 12 or 16.

In another embodiment, the present invention provides a composition as defined above, wherein
—X— is —O-n-butylene-O—;
n is 40 or 48, and
m is 12 or 16.

In another embodiment, the present invention provides a composition as defined above, wherein
—X— is —O-n-butylene-O—
n is 40, and
m is 16.

In another embodiment, the present invention provides a composition as defined above, wherein
—X— is —O-n-butylene-O—
n is 48, and
m is 12.

In another embodiment, the present invention provides a composition as defined above, wherein
—X— is

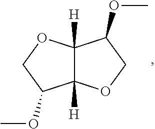

n is 68, and
m is 11

The compounds of formula (I), including reference compounds used herein with e.g. different values for “m” and “n”, are sometimes also designated as “butronic(s)”. In one embodiment, the compound of formula (I) has the more specific formula (I-a)

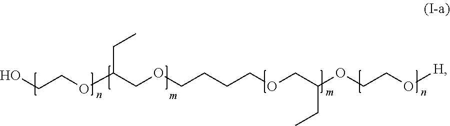

wherein “m” and “n” have the meaning as given for formula (I). In one embodiment, in formula (I-a), m is 12 and n is 48.

The compounds of formula (I) and (I-a) are polymers and can be generally obtained by methods known to the skilled person. The skilled person is aware that, due to the chemical synthesis involved in the manufacture of the compounds of formula (I), the values given for “m” and “n” above are part of a range. In one embodiment, the specific values given for “m” and “n” above represent the prevalent number within that range. In another embodiment, the specific values given for “m” and “n” herein can deviate by up to 4 and up to 6, respectively. For example, in formula (I) or (I-a), when —X— is —O-n-butylene-O—, n is 48±6, or ±5, or ±4, or ±3, or ±2, or ±1; and m is 12±4, or ±3, or ±2, or ±1. In still another embodiment, the weight % (% (w/w)) of the ethoxy moiety (—OE) in the compounds of formula (I) or (I-a) is between 60 to 80% (w/w), or between 60 to 75% (w/w), or between 60 to 70% (w/w).

Moreover, and consistent with the ranges for “m” and “n”, as explained above, the skilled person knows that the synthesis of compounds of formula (I) or (I-a) can lead to a mixture of products within a range of molecular weights (MW). In one embodiment, the specific molecular weight indicated herein, for example in Table 1, is the molecular weight of the predominant product in that mixture. In another embodiment, compounds of formula (I) or (I-a) have a molecular weight in the range between 4000 to 10000 g/mol; or 4000 to 7000 g/mol; or 5000 to 7000 g/mol; or 5500 to 6500 g/mol; or 5900 to 6100 g/mol. In yet another embodiment, the compounds of formula (I) or (I-a) have a molecular weight in the range of 5900 to 6100 g/mol and a weight % of ethoxy units in the range of 60-70% (w/w).

The term “aqueous pharmaceutical composition” means an aqueous composition, formulation or dosage form for pharmaceutical use. In one embodiment said liquid pharmaceutical compositions are for parenteral application of therapeutic antibodies. In another embodiment, the liquid pharmaceutical compositions in accordance with the present invention comprise one or more therapeutic antibodies together with pharmaceutically acceptable excipients or carriers. Such excipients are generally known to a person of skill in the art.

The term “excipient” has its ordinary meaning known to a person of skill in the art of parenteral antibody compositions. In one embodiment, the term “excipient” refers to an ingredient in a pharmaceutical composition or formulation, other than an active ingredient, which is nontoxic to a subject. An excipient includes, but is not limited to, a buffer, stabilizer including antioxidant, or preservative.

The term “pharmaceutical composition” refers to a preparation, formulation or dosage form which is in such form as to permit the biological activity of an active ingredient contained therein to be effective, and which contains no additional components which are unacceptably toxic to a subject to which the pharmaceutical composition would be administered.

The term “pharmaceutically acceptable carrier” refers to an ingredient in a pharmaceutical composition or formulation, other than an active ingredient, which is nontoxic to a subject. A pharmaceutically acceptable carrier includes, but is not limited to an excipient as defined herein.

The term “buffer” is well known to a person of skill in the art of organic chemistry or pharmaceutical sciences such as, for example, pharmaceutical preparation development. Buffer as used herein means acetate, succinate, citrate, arginine, histidine, phosphate, Tris, glycine, aspartate, and glutamate buffer systems. The pH range provided by said buffer is from 4 to 8, preferably from 4.5 to 7.5, more preferably from 5 to 7. Furthermore, within this embodiment, the histidine concentration of said buffer is from 5 to 50 mM, preferably from 10 to 25 mM.

The term “stabilizer” is well known to a person of skill in the art of organic chemistry or pharmaceutical sciences such as, for example, pharmaceutical preparation development. A stabilizer in accordance with the present invention is selected from the group consisting of sugars, sugar alcohols, sugar derivatives, or amino acids. In one aspect the stabilizer is selected from one or several of the following groups (1) sucrose, trehalose, cyclodextrines, sorbitol, mannitol, glycine, or/and (2) methionine, and/or (3) arginine, or lysine. In one embodiment said stabilizers can be used in a concentration up to 500 mM, or up to 350 mM, or up to 250 mM, or up to and including 150 mM. In still another embodiment, the concentration of said stabilizer is for group (1) up to 500 mM, or up to 350 mM, or up to 250 mM, or up to and including 150 mM; for group (2) from 5 to 40 mM, or from 5 to 30 mM, or from 5 to 25 mM; or/and for group (3) up to 350 mM, or up to 250 mM.

The term “antibody” herein is used in the broadest sense and encompasses various antibody classes or structures, including but not limited to monoclonal antibodies, multispecific antibodies (e.g., bispecific antibodies), antibody-cytokine fusion proteins (fpMab's), and antibody fragments so long as they exhibit the desired antigen-binding activity. In one embodiment the fusion protein in the antibody-cytokine fusion proteins is IL-2. In one embodiment, the term “antibody” as used herein refers to multimeric proteins, preferably pentameric proteins.

In one embodiment in accordance with the present invention the antibody is a monoclonal antibody. The term “monoclonal antibody” is known to a person of skill in the art. In one embodiment, the term “monoclonal antibody” refers to an antibody that is derived from a single clone, including any eukaryotic, prokaryotic, or phage clone, and not the method by which it is produced.

In one aspect, the antibody is an “antibody product” selected from alemtuzumab (LEMTRADA®), atezolizumab (TECENTRIQ®), bevacizumab (AVASTIN®), cetuximab (ERBITUX®), panitumumab (VECTIBIX®), pertuzumab (PERJETA®, 2C4, Omnitarg), trastuzumab (HERCEPTIN®), tositumomab (Bexxar®), abciximab (REOPRO®), adalimumab (HUMIRA®), apolizumab, aselizumab, atlizumab, bapineuzumab, basiliximab (SIMULECT®), bavituximab, belimumab (BENLYSTA®) briankinumab, canakinumab (ILARIS®), cedelizumab, certolizumab pegol (CIMZIA®), cidfusituzumab, cidtuzumab, cixutumumab, clazakizumab, crenezumab, daclizumab (ZENAPAX®), dalotuzumab, denosumab (PROLIA®, XGEVA®), eculizumab (SOLIRIS®), efalizumab, epratuzumab, erlizumab, emicizumab (HEMLIBRA®), felvizumab, fontolizumab, gantenerumab, golimumab (SIMPONI®), ipilimumab, imgatuzumab, infliximab (REMICADE®), labetuzumab, lebrikizumab, lexatumumab, lintuzumab, lucatumumab, lulizumab pegol, lumretuzumab, mapatumumab, matuzumab, mepolizumab, mogamulizumab, motavizumab, motovizumab, muronomab, natalizumab (TYSABRI®), necitumumab (PORTRAZZA®), nimotuzumab (THERACIM®), nolovizumab, numavizumab, obinutuzumab (GAZYVA®), olokizumab, omalizumab (XOLAIR®), onartuzumab (also known as MetMAb), palivizumab (SYNAGIS®), pascolizumab, pecfusituzumab, pectuzumab, pembrolizumab (KEYTRUDA®), pexelizumab, priliximab, ralivizumab, ranibizumab (LUCENTIS®), reslivizumab, reslizumab, resyvizumab, robatumumab, rontalizumab, rovelizumab, ruplizumab, sarilumab, secukinumab, seribantumab, sifalimumab, sibrotuzumab, siltuximab (SYLVANT®) siplizumab, sontuzumab, tadocizumab, talizumab, tefibazumab, tocilizumab (ACTEMRA®), toralizumab, tucusituzumab, umavizumab, urtoxazumab, ustekinumab (STELARA®), vedolizumab (ENTYVIO®), visilizumab, zanolimumab, zalutumumab.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. In certain aspects, the antibody is of the IgG1 isotype. In certain aspects, the antibody is of the IgG1 isotype with the P329G, L234A and L235A mutation to reduce Fc-region effector function. In other aspects, the antibody is of the IgG2 isotype. In certain aspects, the antibody is of the IgG4 isotype with the S228P mutation in the hinge region to improve stability of IgG4 antibody. The heavy chain constant domains that correspond to the different classes of immunoglobulins are called a, d, e, g, and m, respectively. The light chain of an antibody may be assigned to one of two types, called kappa (κ) and lambda (λ), based on the amino acid sequence of its constant domain. In one embodiment, the antibody in accordance with the present invention is an IgG 1 and/or IgG4 antibody.

In one embodiment, any of the antibodies in accordance with the present invention is human or humanized. A “human antibody” is one which possesses an amino acid sequence which corresponds to that of an antibody produced by a human or a human cell or derived from a non-human source that utilizes human antibody repertoires or other human antibody-encoding sequences. The human antibodies of the invention may include amino acid residues not encoded by human germline immunoglobulin sequences (e.g., mutations introduced by random or site-specific mutagenesis in vitro or by somatic mutation in vivo).

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

In one embodiment, the present invention provides the compositions as defined herein, wherein the antibody is present at a concentration which provides its desired pharmaceutical activity and an acceptable safety profile. In another embodiment, the present invention provides the compositions as defined herein, wherein the antibody is present at a concentration range from 1 to 220 mg/ml, preferably 5 to 180 mg/ml, or 5 to 100, or 5 to 25 mg/ml.

In another embodiment, the present invention provides the compositions as defined herein, wherein the surfactant is present at a concentration of 0.001 to 1.0 mg/ml; or 0.01 to 1.0 mg/ml, or 0.06 to 1.0 mg/ml; or 0.06 to 0.6 mg/ml.

In another embodiment, the present invention provides the compositions as defined herein, further comprising additional pharmaceutically acceptable excipients.

In another embodiment, the present invention provides a compound of formula (I)

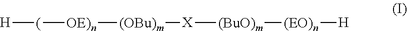

for use as surfactant in aqueous antibody compositions, wherein
—OE is ethoxy;
—OBu is n-butoxy;
—X— is —O-n-butylene-O— or

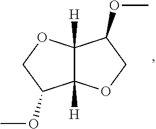

n is 40, 48 or 68 and
m is 11, 12 or 16.

In yet another embodiment, the present invention provides the compound of formula (I) for use as defined above, i.e. as surfactant in aqueous antibody compositions, wherein said compound stabilizes the antibody against aggregation. In one embodiment said aggregation is an aggregation of several antibodies. In another embodiment said aggregation is an aggregation of antibodies and PDMS.

In another embodiment, the present invention provides the compound of formula (I) for use as defined above, wherein said compound prevents the formation of visible particles in an aqueous antibody composition.

In another embodiment, the present invention provides the compound of formula (I) for any use as defined herein before, wherein
—X— is —O-n-butylene-O—;
n is 40 or 48, and
m is 12 or 16.

In another embodiment, the present invention provides the compound of formula (I) for any use as defined herein before, wherein
—X— is —O-n-butylene-O—
n is 40, and
m is 16.

In another embodiment, the present invention provides the compound of formula (I) for any use as defined herein before, wherein
—X— is —O-n-butylene-O—
n is 48, and
m is 12.

In another embodiment, the present invention provides the compound of formula (I) for any use as defined herein before, wherein
—X— is

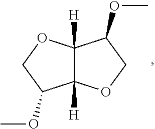

n is 68, and
m is 11

In another embodiment, the present invention provides the compound of formula (I) for any use as defined herein before, wherein the antibody is a monoclonal antibody.

In another embodiment, the present invention provides the compound of formula (I) for any use as defined herein before, wherein the monoclonal antibody is of the IgG1—or IgG4 subclass.

In another embodiment, the present invention provides the compound of formula (I) for any use as defined herein before, wherein the surfactant is present at a concentration of 0.001 to 1.0 mg/ml; or 0.01 to 1.0 mg/ml, or 0.06 to 1.0 mg/ml; or 0.06 to 0.6 mg/ml.

In another embodiment, the present invention provides the compound of formula (I) for any use as defined herein before, further comprising additional pharmaceutically acceptable excipients.

In yet another embodiment, the present invention provides a method to prevent the formation of visible particles in an aqueous antibody composition, said method comprising the use of a compound of formula (I)

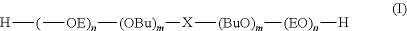

wherein
—OE is ethoxy;
—OBu is n-butoxy;
—X— is —O-n-butylene-O— or

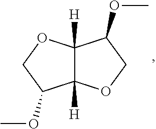

n is 40, 48 or 68, and
m is 11, 12 or 16.

In yet another embodiment, the present invention provides a method to prevent the formation of visible particles in an aqueous antibody composition, said method comprising the use of a compound of formula (I) as defined herein before, wherein
—X— is —O-n-butylene-O—;
n is 40 or 48, and
m is 12 or 16.

In yet another embodiment, the present invention provides a method to prevent the formation of visible particles in an aqueous antibody composition, said method comprising the use of a compound of formula (I) as defined herein before, wherein
—X— is —O-n-butylene-O—
n is 40, and
m is 16.

In yet another embodiment, the present invention provides a method to prevent the formation of visible particles in an aqueous antibody composition, said method comprising the use of a compound of formula (I) as defined herein before, wherein
—X— is —O-n-butylene-O—
n is 48, and
m is 12.

In yet another embodiment, the present invention provides a method to prevent the formation of visible particles in an aqueous antibody composition, said method comprising the use of a compound of formula (I) as defined herein before, wherein
—X— is

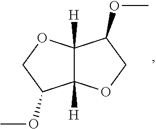

n is 68, and
m is 11

In yet another embodiment, the present invention provides a method to prevent the formation of visible particles in an aqueous antibody composition, said method comprising the use of a compound of formula (I) as defined herein before, wherein the antibody is a monoclonal antibody.

In yet another embodiment, the present invention provides a method to prevent the formation of visible particles in an aqueous antibody composition, said method comprising the use of a compound of formula (I) as defined herein before, wherein the monoclonal antibody is of the IgG1—or IgG4 subclass.

In yet another embodiment, the present invention provides a method to prevent the formation of visible particles in an aqueous antibody composition, said method comprising the use of a compound of formula (I) as defined herein before, wherein the compound of formula (I) is present at a concentration of 0.001 to 1.0 mg/ml; or 0.01 to 1.0 mg/ml, or 0.06 to 1.0 mg/ml; or 0.06 to 0.6 mg/ml.

In yet another embodiment, the present invention provides a method to prevent the formation of visible particles in an aqueous antibody composition, said method comprising the use of a compound of formula (I) as defined herein before further comprising additional pharmaceutically acceptable excipients.

The uses and methods of the present invention are suitable to prevent the formation of visible particles in an aqueous antibody composition. In one embodiment, the formation of visible particles occurs upon storage of said aqueous antibody compositions. The term “storage” as used herein means keeping an aqueous pharmaceutical preparation under conditions known to a person of skill in the art, or as for example indicated in the package inserts of comparable commercially available drugs, or their corresponding Summary of Product Characteristics. In one aspect said storage involves a time of up to 6 months, or 12 months, or 18 months, or 24 months, or 30 months. In another aspect said storage involves keeping said liquid pharmaceutical composition up to its shelf life as approved by regulatory authorities under conditions (such as e.g. temperature) as also approved by such regulatory authority. In one aspect such shelf life and storage conditions can, for example, be found in the package insert accompanying an approved protein based drug, or the corresponding Summary of Product Characteristics. In another aspect the storage temperature for any of the approved storage times is below 30° C. In another aspect the storage temperature is from 2-30° C. In yet another aspect the storage temperature is from 2-8° C.

A set of clauses defining the invention and its preferred aspects and embodiments is as follows:
1. An aqueous pharmaceutical composition comprising an antibody and a surfactant wherein the surfactant is a compound of formula (I)

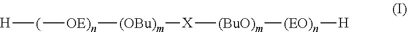

wherein
   —OE is ethoxy;
   —OBu is n-butoxy;
   —X— is —O-n-butylene-O— or

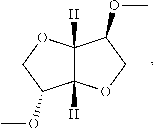

n is 40, 48 or 68, and
m is 11, 12 or 16.
2. The composition according to clause 1, wherein
   —X— is —O-n-butylene-O—;
   n is 40 or 48, and
   m is 12 or 16.
3. The composition according to clause 1, wherein
   —X— is

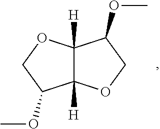

n is 68, and
m is 11
4. The composition according to any one of clause 1 to 3, wherein the antibody is a monoclonal antibody.
5. The composition according to clause 4 wherein the monoclonal antibody is of the IgG1—or IgG4 subclass.
6. The composition according to any one of clause 1 to 5, wherein the surfactant is present at a concentration of 0.001 to 1.0 mg/ml; or 0.01 to 1.0 mg/ml, or 0.06 to 1.0 mg/ml; or 0.06 to 0.6 mg/ml.
7. The composition according to any one of clause 1 to 6 further comprising additional pharmaceutically acceptable excipients.
8. A compound of formula (I)

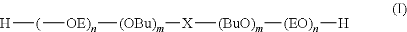

for use as surfactant in aqueous antibody compositions, wherein
   —OE is ethoxy;
   —OBu is n-butoxy;
   —X— is —O-n-butylene-O— or

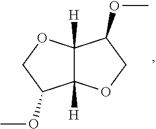

n is 40, 48 or 68, and
m is 11, 12 or 16.
9. The compound of formula (I) for use according to clause 8, wherein said compound stabilizes the antibody against aggregation.
10. The compound of formula (I) for use according to clause 9, wherein said aggregation is either an aggregation of several antibodies or an aggregation of antibodies and PDMS.
11. The compound of formula (I) for use according to clause 8, wherein said compound prevents the formation of visible particles in an aqueous antibody composition.
12. The compound of formula (I) for use according to any one of clauses 8 to 11, wherein
   —X— is —O-n-butylene-O—;
   n is 40 or 48, and
   m is 12 or 16.
13. The compound of formula (I) for use according to any one of clauses 8 to 11, wherein
   —X— is

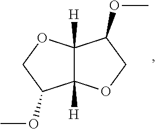

n is 68, and
m is 11
14. The compound of formula (I) for use according to any one of clauses 8 to 13, wherein the antibody is a monoclonal antibody.
15. The compound of formula (I) for use according to clause 14, wherein the monoclonal antibody is of the IgG1—or IgG4 subclass.
16. The compound of formula (I) for use according to any one of clauses 8 or 15, wherein the surfactant is present at a concentration of 0.001 to 1.0 mg/ml; or 0.01 to 1.0 mg/ml, or 0.06 to 1.0 mg/ml; or 0.06 to 0.6 mg/ml.
17. The compound of formula (I) for use according to any one of clauses 8 to 16 further comprising additional pharmaceutically acceptable excipients.
18. A method to prevent the formation of visible particles in an aqueous antibody composition, said method comprising the use of a compound of formula (I)

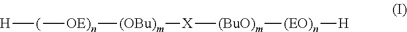

wherein
   —OE is ethoxy;
   —OBu is n-butoxy;
   —X— is —O-n-butylene-O— or

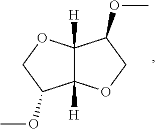

n is 40, 48 or 68, and
m is 11, 12 or 16.
19. The method according to clause 18, wherein
   —X— is —O-n-butylene-O—;
   n is 40 or 48, and
   m is 12 or 16.
20. The method according to clause 18, wherein
   —X— is

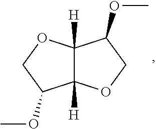

n is 68, and
m is 11
21. The method according to any one of clauses 18 to 20, wherein the antibody is a monoclonal antibody.
22. The method according to clause 21 wherein the monoclonal antibody is of the IgG1—or IgG4 subclass.
23. The method according to any one of clauses 18 to 22, wherein the compound of formula (I) is present at a concentration of 0.001 to 1.0 mg/ml; or 0.01 to 1.0 mg/ml, or 0.06 to 1.0 mg/ml; or 0.06 to 0.6 mg/ml.
24. The method according to any one of clauses 18 to 23 further comprising additional pharmaceutically acceptable excipients.
25. The compositions, uses and methods according to any one of clauses 1 to 24, wherein the compound of formula (I) has the more specific formula (I-a) as defined herein.

The invention will now be further illustrated by the following, non-limiting working examples

### EXAMPLES

### Materials and Methods

### Materials

The model antibodies (mAb), including a pentameric protein, used for this study were provided by F. Hoffmann-La Roche (Basel, Switzerland):

Glycoengineered IgG1 mab (gMab) formulated in 20 mM His-HCl buffer (Ajinomoto, Tokyo, Japan) with 240 mM trehalose (Pfanstiehl Inc., Illinois, USA) at pH 6.0

IgG1 mab 1 (Mab1) formulated in 10 mM His-HCl buffer (Ajinomoto, Tokyo, Japan) with 240 mM sucrose (Pfanstiehl Inc., Illinois, USA) at pH 6.0.

IgG1 mAb 2 (Mab2) formulated in 20 mM His-HCl buffer (Ajinomoto, Tokyo, Japan) with 200 mM trehalose (Pfanstiehl Inc., Illinois, USA) at pH 5.5.

Bispecific mAb 1 (bsMab1) formulated in 20 mM His-HCl buffer (Ajinomoto, Tokyo, Japan) with 240 mM sucrose (Pfanstiehl Inc., Illinois, USA) at pH 6.0.

Bispecific mAb 2 (bsMab2) formulated in 10 mM His-HCl buffer (Ajinomoto, Tokyo, Japan) with 240 mM sucrose (Pfanstiehl Inc., Illinois, USA) at pH 5.8.

IgG4 mAb 2 (IgG4-2) formulated in 20 mM Histidine buffer (Ajinomoto, Tokyo, Japan) with 200 mM arginine succinate (Ajinomoto, Tokyo, Japan) at pH 5.7.

Multimeric Protein (MP, here pentameric) formulated in 10 mM Na-phosphate buffer (Merck KGAA, Darmstadt, Germany; chem. Fabrik Budenheim, Budenheim, Germany) with 5% (m/v) sorbitol (Merck KGAA, Darmstadt, Germany) at pH 7.5.

Antibody-cytokine fusion Protein mAb (fpMab) formulated in 20 mM His-HCl buffer (Ajinomoto, Tokyo, Japan) with 240 mM sucrose (Pfanstiehl Inc., Illinois, USA) at pH 5.5.

The antibodies were tested in a concentration range of 5 to 180 mg/mL in aqueous buffer solutions.

The screened surfactants were provided by BASF (Ludwigshafen, Germany): Pluronic PE 10400 (Px334), Pluronic PE 10500 (Px335), butronic (Bux016, Bux017), butronic 4060 (Bux164), butronic 6060 (Bux190), butronic 6070 (Bux199), isosorbid alkoxylate (IA80, IA90), Poly (methyl-butyl-methyl) oxazoline (Pz110, Pz120), and Polyvinylalcohole/polypropylenglycole (PVA/PPG). Polysorbate 20 (PS20; Croda International, Snaith, UK) and Poloxamer 188 (Px188; BASF, Ludwigshafen, Germany) were used as guiding references. A summary of the tested compounds, including reference surfactants, is provided in Table 1.

TABLE 1

| | “X” in formula (I) | “m” (in formula (I)) | “n” (in formula (I) | MW |
|---|---|---|---|---|
| Compounds | | | | |
| tested | | | | |
| Bux016 | 1,4 Butandiol | 10 | 17 | 3040 |
| Bux017 | 1,4 Butandiol | 10 | 70 | 7658 |
| Bux164 | 1,4 Butandiol | 10 | 27 | 3911 |
| Bux190 | 1,4 Butandiol | 16 | 40 | 5922 |
| Bux199 | 1,4 Butandiol | 12 | 48 | 6050 |
| IA90 | Isosorbid | 11 | 68 | 7700 |
| IA84 | Isosorbid | 34 (here PO/OH*) | 44 | 7973 |
| Reference | | | | |
| Surfactants | | | | |
| Pz110 | Poly(-Methyl-Buthyl-Methyl)-Oxazolin | 25.6% Bu | 74.4% Me | |
| Pz120 | Poly(-Methyl-Buthyl-Methyl)-Oxazolin | 24.3% Bu | 75.7% Me | |
| PVA/PPG | Polyvinylalkohol/Polypropylenglykol | 80/20 | | |
| Px334 | Pluronic PE 10400 | 40 wt % EO | commercial | 5900 |
| Px335 | Pluronic PE 10500 | 50 wt % EO | commercial | 6500 |

*PO/OH = n-propoxy

All other reagents like methanol (MeOH), potassium dihydrogen phosphate anhydride (KH₂PO₄), dipotassium hydrogen phosphate anhydride (K₂HPO₄), and potassium chloride (KCl) were of analytical grade and obtained from Merck KGa, Darmstadt, Germany.

### Methods

### Evaluation of Thermal Conformational Protein Stability in Presence of Surfactants

Prometheus NT.Plex (NanoTemper Technologies GmbH, München, Germany) was used to investigate conformational protein stability. This device allows the label-free detection of the change in intrinsic protein fluorescence from aromatic tryptophan and tyrosine residues by using only small quantities of solution. Thermal induced protein unfolding was monitored by detecting the emission shift at 330 nm and 350 nm at an optimized laser power of 7-12%. The nanoDSF standard grade capillary chips (NanoTemper Technologies, MUnchen, Germany) were filled with 10 μL of freshly prepared formulation containing 25 mg/mL mAb compounded with either 0.01, 0.1, 1 or 10 mg/mL of the specific surfactant. The analysis was performed with 5 different mAbs (bsMab1, Mab1, gMab, bsMab2, and fpMab). Samples were heated with a constant heating ramp of 0.5° C. per minute from 20 to 95° C. The PR.StabilityAnalysis software (NanoTemper Technologies, München, Germany) automatically calculated the onset (Tₒₙ) and first transition point (Tₘ₁) of the melting curve. Data reported are the mean value of two individual measurements.

Evaluation of Protein Stability in Presence of Surfactants after Mechanical Stresses and Thermal Stability

Surfactant performance screens were carried out with 0.06, 0.2, and 0.6 mg/mL of the surfactant (for prescreen with fpMab: 0.001, 0.01, 0.1, and 1 mg/mL) in the formulations of different model mAbs described within the materials section. All formulations were compounded and the liquid samples were sterile filtered through 0.22 μm Millex Sterivex™ GV (Millipore, Bedford, USA) filter units. Formulations containing fpMab, gMab, bsMab1, Mab1, bsMab2, IgG4, and MP were filled into 6 mL type 1 glass vials and closed with Ø20 mm Teflon® coated serum stoppers (DAIKYO Seiko Ltd., Tokyo, Japan). The stoppered vials were crimped using an aluminum cap with PP-plate (Datwyler Holding AG, Altdorf, Switzerland). Mab1 was additionally filled into 2.25 mL prefillable syringes BD Neopak™ (pfs), Mab2 was filled into 1 mL pfs (both BD Medical—Pharmaceutical Systems, Franklin Lakes, US) and closed with Teflon® coated serum stoppers for 2.25 mL or 1 mL pfs respectively (DAIKYO Seiko Ltd., Tokyo, Japan).

To evaluate the effects of the surfactants on the mAb's stability different interfacial stress conditions like agitation and multiple freeze-thaw cycles were applied to fpMab, bsMab2, IgG4, and MP formulations. Shaking was performed by placing the vials horizontally in a shaker (HS 260 Control Model; IKA Werke GmbH & Co. KG; Staufen, Germany) for seven days at 5° C. and 25° C. protected from light at constant 200 rounds per minute (rpm). Freeze-thaw (F/T) stress was performed by exposing the vials to five consecutive cycles of freezing at −20° C. and thawing at 5° C. controlled by climate chamber VTM 4004 (Vötsch, Borken, D). For MP only F/T stress was tested, for fpMab only shaking for seven days at 5° C.

Thermal stability data was generated by storing the liquid gMab, bsMab1, Mab1, and Mab2 formulations for 9 months (mo) at 5° C., for 6 months at 25° C./60% relative humidity (rH) and for 12 weeks at 40° C./75% rH. Samples were analyzed at initial time point (t0) and after 1 mo, 3 mo, 6 mo, and 9 mo of storage using the subsequently described analytical methods.

### Visible Particles (VP)

Visual inspection was performed as previously described using a black-and-with-box (Color Viewing Light 3 BASIC, JUST Normlicht, Weilheim, D) according to Ph. Eur. 2.9.20 [1]. Vials and pfs' designated for visual inspection are analyzed after equilibrating at RT. Vials and pfs' are stored again after inspection and the same vials are used for visual inspection at each time point. The number of particles was categorized into four classes: class (I) is equivalent to 0 particles, class (II) is equivalent to 1-4 particles in max 30% vials, class (III) is equivalent to >5 particles in <30% vials/1 particles in <50% vials and class (IV) is equivalent >30% vials >5 particles/>40% vials >2 particles.

### Turbidity (Opalescence and Clarity)

Turbidity was determined as previously described in the literature and according to Ph. Eur. 2.2.1 using a TL 2350 EPA turbidimeter (Hach Lange GmbH, Düsseldorf, Germany) calibrated with a StablCal® calibration kit (Hach Lange GmbH). Results were given as Nephelometric Turbidity Units (NTU). [2, 3]

### Light Obscuration

Sub-visible particle (SVP) count was measured by means of light obscuration using a HIAC 9703+ liquid particle counting system (Beckman Coulter, Pasadena, US) and PharmSpec 3 (Hach Lange GmbH) software. The applied measurement technique was adapted from the method described in Ph.Eur. 2.9.19 [4] and USP <787> [5]. After rinsing the system with sample solution, four runs with a sample volume of 0.2 mL were performed. The final cumulative particle count was obtained by calculating the mean±SD (standard deviation) from the last three measurements. SVP bigger than or equal to 2, 5, 10, 25 and 50 μm were detected and presented as cumulative counts per mL of solution.

### Backgrounded Membrane Imaging

SVP count of liquid samples was also measured by Backgrounded Membrane Imaging (BMI) technology using a Horizon system and Halo Lab software (both from Halo Lab, Burlingame, USA). A polycarbonate membrane plate with pore size of 0.4 um (Halo Lab, Burlingame, USA) is first washed with particle free water. The washed filter is filled with 40 μL of sample (2 min vacuum of 200 mbar) and measured. The final cumulative particle count was obtained by calculating the mean±SD from three individual measurements. SVP bigger than or equal to 2, 5, 10, 25 and 50 μm were detected and presented as cumulative counts per mL of solution.

### Size-Exclusion (Ultra) High Performance Chromatography (SE-(U)HPLC)

Soluble mAb aggregates, in the following referred to as high molecular weight species (HMWs), the monomer and the low molecular weight species (LMWs) were analyzed by SE-HPLC for gMab, bsMab1, Mab1, and Mab2 or analyzed by SE-UHPLC for bsMab2, IgG4, and MP.

SE-HPLC: The system used consisted of an Alliance 2695 HPLC instrument equipped with a 2489 UV detector (both from Waters Corporation, Milford, MA). The autosampler temperature was set to 5° C. and the column was loaded with total 150 μg of the mAb. Separation was performed using a TSK G3000 SWXL, 7.8×300 mm column (Tosoh Bioscience, Stuttgart, Germany) at a constant oven temperature of 25° C. As mobile phase a buffer containing 200 mM K₂HPO₄/KH₂PO₄ and 250 mM KCl pH 7.0 was used with a flow rate of 0.5 mL/min. Signal detection was executed at a wavelength of 280 nm and the Empower 3 Chromatography Data System software (Waters Corporation, Milford, MA) was used to calculate the peak area percent.

SE-UHPLC: The utilized system comprised an Thermo UltiMate 3000 UHPLC instrument equipped with a 3000 UV/vis detector (both from Thermo Fisher Scientific, Waltham, US). The autosampler temperature was set to 10° C. and the system was loaded with total 50 μg of the mAb. Separation was performed using a TSK UP-SW3000, 4.6×300 mm column (Tosoh Bioscience, Stuttgart, Germany) at a constant oven temperature of 25° C. and a mobile phase of 200 mM K₂HPO₄/KH₂PO₄ and 250 mM KCl pH 6.2 at a flow rate of 0.3 mL/min. Signal detection was executed at a wavelength of 280 nm and the Empower 3 Chromatography Data System software (Waters Corporation, Milford, MA) was used to calculate the peak area percent.

### Particle Identification (FTIR)

Particle identification was performed by FTIR microscopy using Nicolet iN10 FT-IR microscope (Thermo Fisher Scientific Inc., Massachusetts, USA). First, the sample was filtered by gold-coated polycarbonate filters (Unchained Labs, Pleasanton, USA) with a pore size of 0.8 μm and a filtration area diameter of 4 mm. Filter conditioning includes a few droplets of 0.22 μm filtrated ethanol to open the pores followed by filtration of approximately 1 mL of particle-free water as a washing step. The full content of each vial (cooled in a cold bath before) was poured directly on the filter surface. As a final step, each filter was washed with cooled particle-free water. FTIR analysis of particles and areas of non-defined particles in the filter surface was performed by applying the microscope reflection mode. Particle nature was defined by spectra comparison with internal and commercial libraries.

### Flow Imaging (FlowCam)

Particle morphology was characterized by a flow imaging technique using a FlowCam 8000 instrument (Fluid Imaging Technologies Inc., Scarborough, USA) with a 300 μm flow cell and 4× magnification. Prior to each measurement, the system was pre-rinsed with sample solution. Samples were analyzed with a sampling efficiency of 75% and a flow rate of 2 mL/min.

### Example 1: Prescreen—Evaluation of Thermal Conformational Protein Stability in Presence of Surfactants

Maximization of the conformational stability is reported to increase long-term drug product quality and/or stability by preventing unfolding and aggregation of therapeutic proteins [6]. High throughput and low volume screening techniques are DSC (Differential Scanning Calorimetry) or measuring the intrinsic protein fluorescence under isothermal chemical denaturation (ICD) or thermal denaturation conditions by means of nanoDSF (Differential Scanning Fluorimetry) [7]. To exclude a negative impact of the surfactants on the protein's conformational stability thermal DSF measurements were performed.

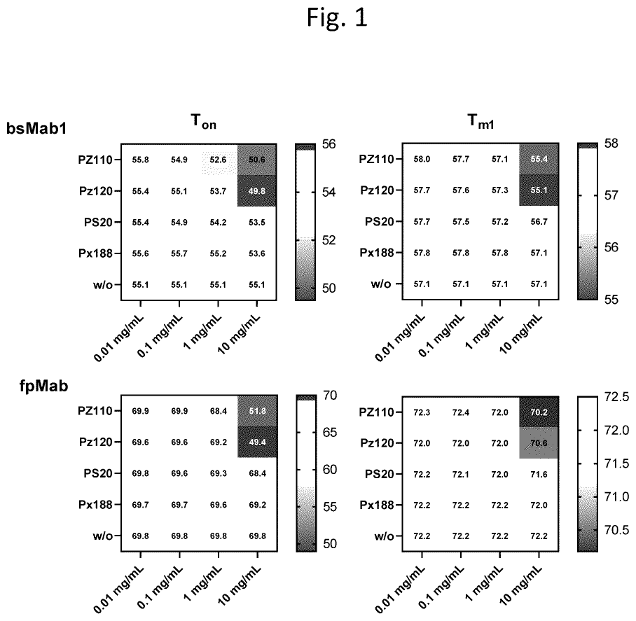

The onset temperature (Tₒₙ) of unfolding and the first melting transition (Tₘ₁) were measured as stability indicating parameters for five different mAbs (bsMab1, Mab1, gMab, bsMab2, fpMab) in presence of surfactants at concentrations ranging from 0.01 mg/mL to 10 mg/mL. The values of the mAb without added surfactant and with PS20 and Px188 were taken as references. In general, the majority of the conditions tested did not show any significant effect on the mAbs' conformational stability. Data of formulations with considerable changes in their transition and melting temperature were presented as a heat map (FIG. 1): the darker the color, the stronger the decrease in the respective temperature due to the presence of the surfactant.

All surfactants from the chemical groups poloxamer, butronic, isosorbide alkoxylate, and PVA/PPG did not show any effect on the tested mAbs in the tested concentration range. However, both tested surfactants of the polyoxazoline group showed a destabilizing effect of fpMab and bsMab1 at a concentration of 10 mg/mL compared to the reference formulations in both Tₒₙ and Tₘ₁.

### Example 2: Prescreen—Shaking Study

The ability to protect mAbs against mechanical/interfacial stresses was tested by performing a horizontal shaking study with a broad range of surfactant concentration (0.001-1 mg/mL) and measured by visible inspection for visible particles (VP). Based on the data obtained by visual inspections, the formulations were categorized into 4 classes (I-IV) and presented by a heat map. A high number of VPs results in higher class and are presented with darker intensity of grey. Formulations containing either no surfactant, PS20 or Px188 were used as guiding references to assess the performance of the novel surfactants.

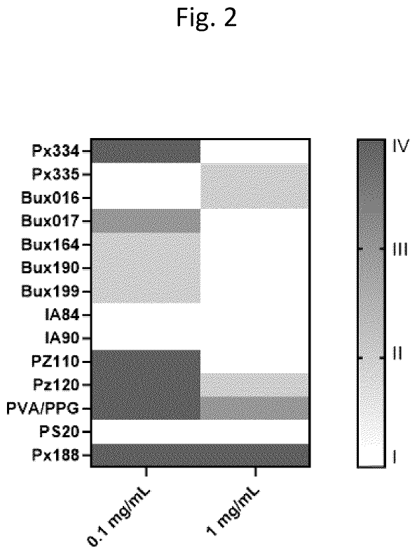

None of the tested surfactants was capable of protecting the mAb (fpMab) against shaking stress in a concentration range of 0.001-0.01 mg/mL. For the ease of reading, only formulations with 0.1 and 1 mg/mL surfactant are shown (FIG. 2).

Most of the surfactants showed good results at 1 mg/mL concentration. Here, only PVA/PPG showed a highly increased number of VP, whereas Px335, Bux016, and Pz120 showed slightly increased numbers. 0.1 mg/mL of Px334, Pz110, Pz120, and PVA/PPG (class IV), as well as 0.1 mg/mL of Bux017 (class III) was insufficient to stabilize the formulations. Also Bux164, Bux190, Bux199, and IA84 showed slightly increased numbers of VP compared to PS20. Px188 could not protect the formulations for shaking stress in all concentrations.

### Example 3: Evaluation of Protein Stability in Presence of Surfactants after Long-Time Storage and Thermal Stress

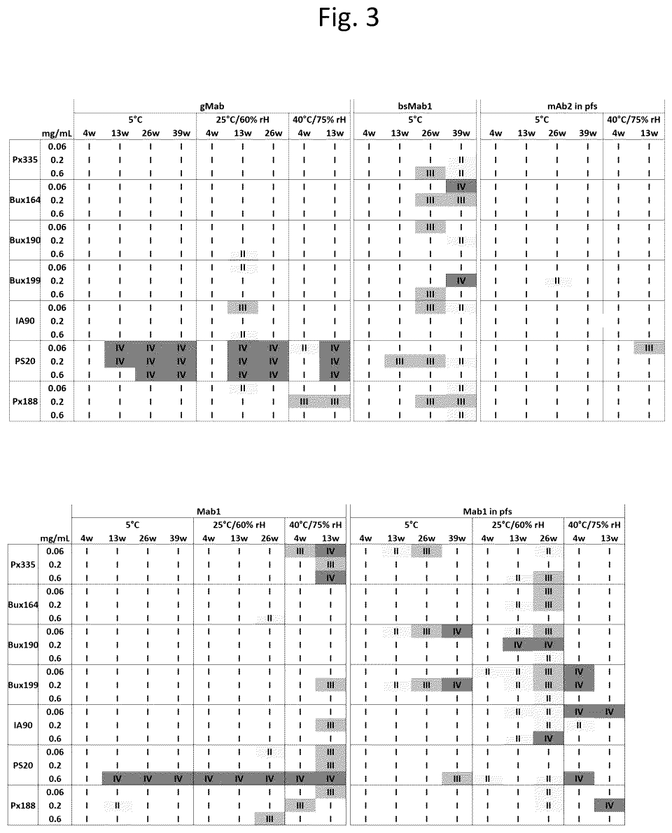

Based on the data obtained in the prescreen studies, a follow-up study was conducted including the five most promising novel surfactant candidates Px335, Bux164, Bux190, Bux199, and IA90. Under long-term storage conditions a potential negative impact on protein stability should have been excluded. Therefore, stability of the formulations with four mAbs (gMab, bsMab1, Mab1, and Mab2) was evaluated over 9 months storage at 5° C., 6 month at 25° C./60% rH, and 3 month at 40° C./75% rH (as described in methods) in terms of formation of visible and sub-visible particles as well as HMWs (FIG. 3-6). Surfactant levels studied were kept constant at 0.06, 0.2, and 0.6 mg/mL, PS20 or Px188 were used as guiding references.

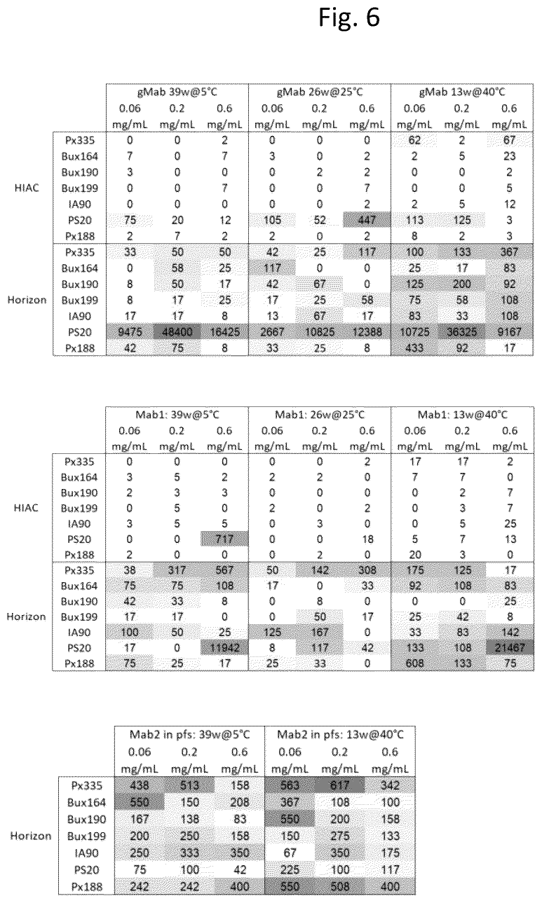

In summary, all novel surfactants have performed better or comparable to the tested references. However, there were slight differences in terms of preventing visible particle formation (FIG. 3). IA90, Bux190 and Bux199 showed a good performance with comparable or better results than PS20 and Px188, whereas Px335 and Bux164 did not. In some PS20 containing formulations, particularly in gMab and Mab1 samples, an increase in visible particles was observed, which was most probably caused by accelerated PS20 degradation and a significant release of free fatty acids. Sub-visible particle counts measured by light obscuration (FIG. 6) were in general at low levels and the reported values are significantly lower than the maximum numbers accepted according to USP <787> and Ph.Eur. 2.9.19. gMab and Mab1 formulations including PS20 showed an increased amount of particles which, based on FlowCam images, are likely to be identified as free fatty acid particles (data not shown). The Backgrounded Membrane Imaging results detected by Horizon (FIG. 6) showed slightly higher SVP counts for Px335 and Bux164 but lower counts for Bux190, Bux199 and IA90 compared to Px188.

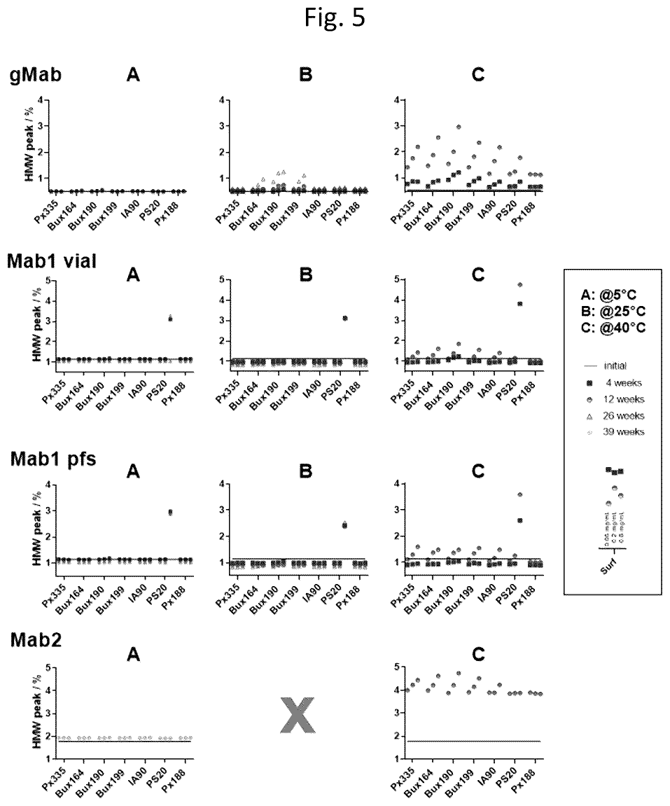

The HMW species by means of SE-HPLC (FIG. 5) showed also no significant change compared to the initial data. A slight increase of HMW content was detected for all butronics after 6 month at 25° C. in gMab formulations, after 3 month at 40° C. in gMab formulations with all novel surfactants and PS20, and in Mab2 formulations with Px335 and all butronics. This increase in HMW species was depending on surfactant content: the higher the concentration, the stronger is the increase.

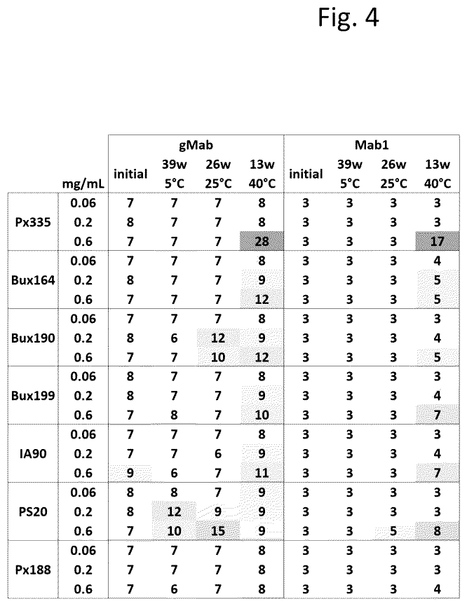

For the turbidity (FIG. 4), it has to be noted that most formulations did not show any considerable changes during these tests. Only the highest concentration of the novel surfactants showed a slight increase of turbidity after 3 months at 40° C. and Px335 a strong increase after 3 months at 40° C. compared to PS20 and Px188.

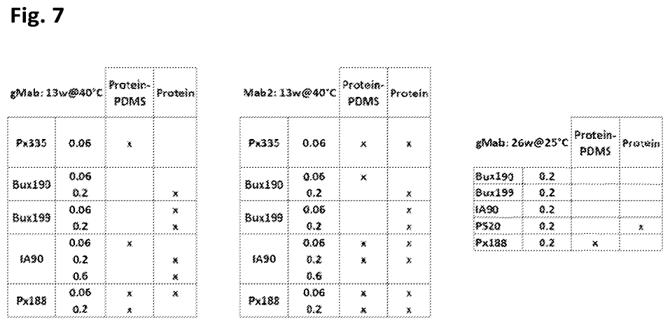

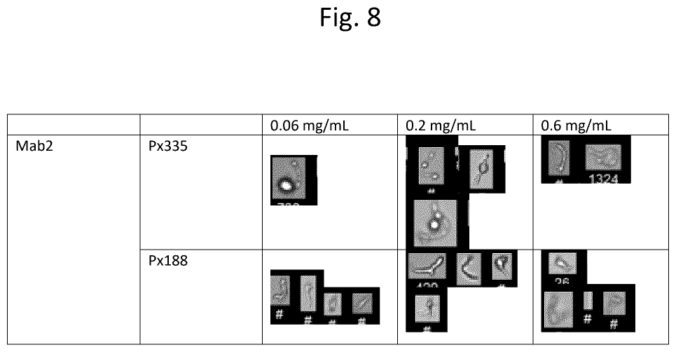

One of the most important challenges in the current use of Px188 is the formation of protein-PDMS particles (PPP). To rule this out, FTIR measurements were carried out with two different mAbs which are known to be prone to PPP formation when formulated with Px188 (gMab and Mab2) on selected samples (FIG. 7). No PPPs were detected in Bux190 and Bux199 formulations 0.2 mg/mL and IA90 formulations 0.6 mg/mL. Both Px188 at 0.06 and 0.2 mg/mL and Px335 at 0.06 mg/mL showed PPP in our FTIR tests. For both Px188 and Px335 PPP were presumably also detect by FlowCam (FIG. 8).

Including all the data from the stability study, Bux190 and IA90 performed best, followed closely by Bux199.

### Example 4: Evaluation of Protein Stability in Presence of Surfactants after Mechanical Stress

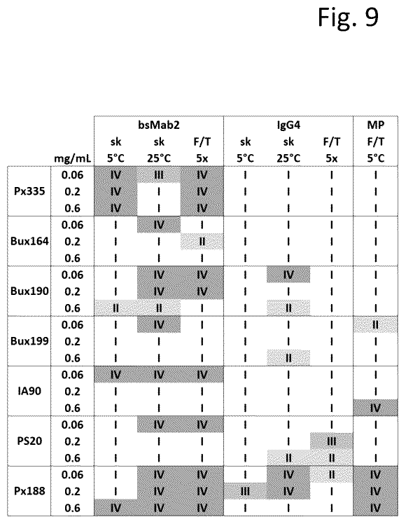

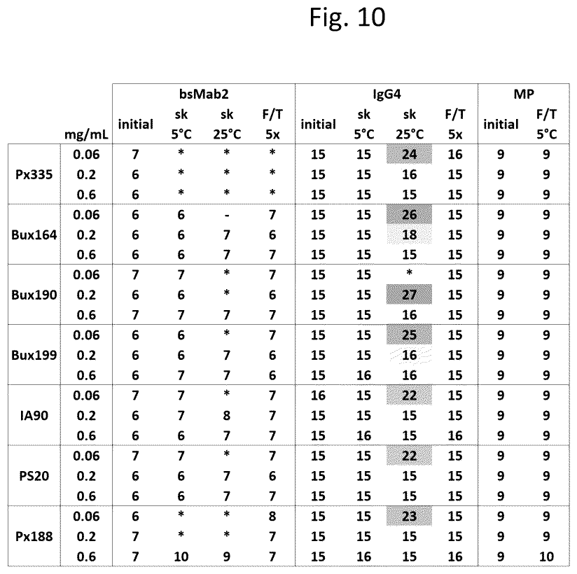

The impact of mechanical/interfacial stress on the mAbs' stability was tested performing agitation and freeze-thaw studies including 3 different active pharmaceutical ingredients (bsMab2, IgG4, and MP). Surfactant levels studied were kept constant at 0.06, 0.2, and 0.6 mg/mL, PS20 or Px188 were used as guiding references. Stability data obtained in terms of formation of visible and sub-visible particles, changes in turbidity as well as HMW species (FIG. 9-12).

All surfactants including references showed insufficient protection of mAbs against visible particle formation at low concentrations (0.06 mg/mL) especially at 25° C. (FIG. 9). At higher concentrations ≥0.2 mg/mL, Bux164, Bux199 and IA90 performed better or comparable to PS20. Bux190 and Px335 showed worse results compared to PS20 and only performed slightly better or comparable to Px188.

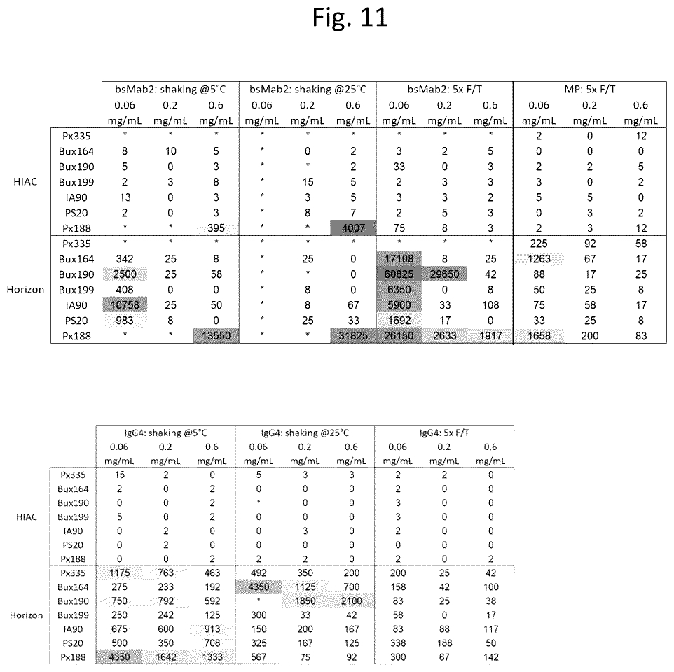

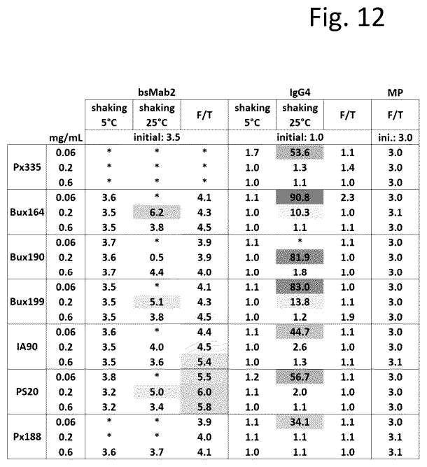

Sub-visible particle counts measured by light obscuration (FIG. 11) were in general at low levels and the reported values were significantly lower than the maximum numbers accepted according to USP <787> and Ph.Eur. 2.9.19. Exceptions were bsMab2 formulations with Px335 in all concentrations, Bux190/Px188≤0.2 mg/mL, Bux164/Bux199/IA90/PS20 ≤0.06 mg/mL, and IgG4 formulation with 0.06 mg/mL Bux190. Here, the particle count was higher than the detection limit. For IgG4 formulations, no strong increase of SVP could be detected but an increase of the turbidity was detected in all formulations with 0.06 mg/mL surfactant and with ≤0.2 mg/mL Bux190. Both, the SVP in bsMab2 and the increase in IgG4 indicate an insufficient stabilizing capacity of the surfactants. The Backgrounded Membrane Imaging results detected by Horizon (FIG. 11) showed all in all higher SVP counts especially for lower surfactant concentrations and Px335. But compared to PS20 also Bux164/Bux190 showed high SVP counts in IgG4 formulations. The monomer contents by means of SE-UHPLC (FIG. 12) showed only minor changes in bsMab2 and MP formulations. For IgG4 a strong increase of HMW species was detected for all surfactants at ≤0.06 mg/mL, for Bux190 also at 0.2 mg/mL, and a slight increase for Bux164 and Bux199 at ≤0.2 mg/mL. Bux199 and IA90 performed really well and highly comparable to PS20.

In summary, the novel surfactants tested here show a good potential to stabilize biologicals compared to the ones currently used. Surfactants from the chemical groups butronic and isosorbide alkoxylate show comparable or better results compared to PS20 and Px188, especially with regard to the prevention of protein PDMS particles. However, the present inventors have demonstrated that even within this class of compounds, significant differences exist as to their ability to act as surfactants and especially as surfactant for stabilizing aqueous antibody compositions.

### ABBREVIATIONS

BMI: Backgrounded Membrane Imaging
Bux: butronic
DSF: Differential Scanning Fluorimetry
F/T: freeze-thaw cycles
FFA: free fatty acid
FTIR: Fourier transform infrared
HMW: high molecular weight species
IA: isosorbide alkoxylate
LMW: low molecular weight species
mAb: monoclonal antibodies
mo: month
NTU: Nephelometric Turbidity Units
PDMS: Polydimethylsiloxane
pfs: pre-fillable syringes
Ph. Eur.: European Pharmacopoeia
PPP: protein-PDMS particles
PS: polysorbate
Px: poloxamer
PZ: polyoxazoline
rH: relative humidity
RT: room temperature
SD: standard derivation
SE-(U)HPLC: Size-exclusion (ultra) high performance chromatography
sk: shaking
Surf: surfactant
SVP: sub-visible particles
Ton: onset temperature
Tm: melting temperature
USP: United States Pharmacopeia
VP: visible particles
w: weeks
w/o: without

References

| | |
|---|---|
| [1] | European Pharmacopeia 11.0, 2.9.20. Particulate contamination: visible particles. The |
| | European Directorate for the Quality of Medicines & Health Care, Strasbourg, France, |
| | 2022. |
| [2] | S. Kiese, A. Papppenberger, W. Friess and H. -C. Mahler, “Shaken, Not Stirred: |
| | Mechanical Stress Testing of an IgG1 Antibody,” J. Pharm. Sci., no. 97, pp. 4347-4366, |
| | 2008. |
| [3] | European Pharmacopeia 11.0, 2.2.1. Clarity and degree of opalescence of liquids. The |
| | European Directorate for the Quality of Medicines & Health Care, Strasbourg, France, |
| | 2022. |
| [4] | European Pharmacopeia 11.0, 2.9.19. Particulate contamination: sub-visible particles. The |
| | European Directorate for the Quality of Medicines & Health Care, Strasbourg, France, |
| | 2022. |
| [5] | “USP <787>, Subvisible particulate matter in therapeutic protein injections, Pharmacopeia |
| | Forum, 38. Pharmacopeia Forum,” 2012. |
| [6] | W. Wang, S. Nema and D. Teagarden, “Protein aggregation-pathways and influencing |
| | factors,” Int. J. Pharm., no. 390, p. 89.99, 2010. |
| [7] | H. Svilenov, U. Markoja and G. Winter, “Isothermal chemical denaturation as a |
| | complementary tool to overcome limitations of thermal differential scanning fluorimetry |
| | in predicting physical stability of protein formulations,” Europ. J. Pharm. and Biopharm., |
| | no. 125, p. 106.113, 2018. |

1. An aqueous pharmaceutical composition comprising an antibody and a surfactant wherein the surfactant is a compound of formula (I) 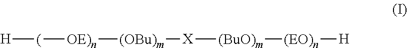
wherein
—OE is ethoxy;
—OBu is n-butoxy;
—X— is —O-n-butylene-O— or 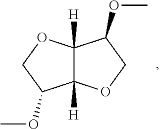
n is 40, 48 or 68, and
m is 11, 12 or 16.

2. The composition according to claim 1, wherein
—X— is —O-n-butylene-O—;
n is 40 or 48, and
m is 12 or 16.

3. The composition according to claim 1, wherein
—X— is 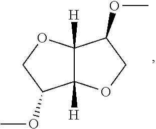
n is 68, and
m is 11.

4. The composition according to claim 1, wherein the antibody is a monoclonal antibody.

5. The composition according to claim 4 wherein the monoclonal antibody is of the IgG1—or IgG4 subclass.

6. The composition according to claim 1, wherein the surfactant is present at a concentration of 0.001 to 1.0 mg/ml.

7. The composition according to claim 1 further comprising additional pharmaceutically acceptable excipients.

8-17. (canceled)

18. A method to prevent the formation of visible particles in an aqueous antibody composition, said method comprising the use of a compound of formula (I) 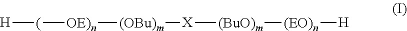
wherein
—OE is ethoxy;
—OBu is n-butoxy;
—X— is —O-n-butylene-O— or 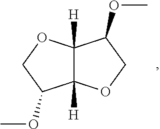
n is 40, 48 or 68, and
m is 11, 12 or 16.

19. The method according to claim 18, wherein
—X— is —O-n-butylene-O—;
n is 40 or 48, and
m is 12 or 16.

20. The method according to claim 18, wherein
—X— is 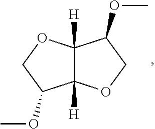
n is 68, and
m is 11.

21. The method according to claim 18, wherein the antibody is a monoclonal antibody.

22. The method according to claim 21 wherein the monoclonal antibody is of the IgG1—or IgG4 subclass.

23. The method according to claim 18, wherein the compound of formula (I) is present at a concentration of 0.001 to 1.0 mg/ml.

24. The method according to claim 18 further comprising additional pharmaceutically acceptable excipients.
